SEQUENCE LISTING (2 sequences)

SEQ ID NO: 1 - DNA, 3419 bp, homo sapien
cccctgcagcggagacagagactgagcggcccggcaccgccatgcctgcgctctggctgggctgctgcctctgcttctcgctcctcctgcccgcagcccgggccacctccaggagggaagtctgtgattgcaatgggaagtccaggcagtgtatctttgatcgggaacttcacagacaaactggtaatggattccgctgcctcaactgcaatgacaacactgatggcattcactgcgagaagtgcaagaatggcttttaccggcacagagaaagggaccgctgtttgccctgcaattgtaactccaaaggttctcttagtgctcgatgtgacaactccggacggtgcagctgtaaaccaggtgtgacaggagccagatgcgaccgatgtctgccaggcttccacatgctcatggatgcggggtgcacccaagaccagagactgctagactccaagtgtgactgtgacccagctggcatcgcagggccctgtgacgcgggccgctgtgtctgcaagccagctgtcactggagaacgctgtgataggtgtcgatcaggttactataatctggatggggggaaccctgagggctgtacccagtgtttctgctatgggcattcagccagctgccgcagctctgcagaatacagtgtccataagatcacctctacctttcatcaagatgttgatggctggaaggctgtccaacgaaatgggtctcctgcaaagctccaatggtcacagcgccatcaagatgtgtttagctcagcccaacgactagaccctgtctattttgtggctcctgccaaatttcttgggaatcaacaggtgagctatggtcaaagcctgtcctttgactaccgtgtggacagaggaggcagacacccatctgcccatgatgtgattctggaaggtgctggtctacggatcacagctcccttgatgccacttggcaagacactgccttgtgggctcaccaagacttacacattcaggttaaatgagcatccaagcaataattggagcccccagctgagttactttgagtatcgaaggttactgcggaatctcacagccctccgcatccgagctacatatggagaatacagtactgggtacattgacaatgtgaccctgatttcagcccgccctgtctctggagccccagcaccctgggttgaacagtgtatatgtcctgttgggtacaaggggcaattctgccaggattgtgcttctggctacaagagagattcagcgagactggggccttttggcacctgtattccttgtaactgtcaagggggaggggcctgtgatccagacacaggagattgttattcaggggatgagaatcctgacattgagtgtgctgactgcccaattggtttctacaacgatccgcacgacccccgcagctgcaagccatgtccctgtcataacgggttcagctgctcagtgatgccggagacggaggaggtggtgtgcaataactgccctcccggggtcaccggtgcccgctgtgagctctgtgctgatggctactttggggacccctttggtgaacatggcccagtgaggccttgtcagccctgtcaatgcaacaacaatgtggaccccagtgcctctgggaattgtgaccggctgacaggcaggtgtttgaagtgtatccacaacacagccggcatctactgcgaccagtgcaaagcaggctacttcggggacccattggctcccaacccagcagacaagtgtcgagcttgcaactgtaaccccatgggctcagagcctgtaggatgtcgaagtgatggcacctgtgtttgcaagccaggatttggtggccccaactgtgagcatggagcattcagctgtccagcttgctataatcaagtgaagattcagatggatcagtttatgcagcagcttcagagaatggaggccctgatttcaaaggctcagggtggtgatggagtagtacctgatacagagctggaaggcaggatgcagcaggctgagcaggcccttcaggacattctgagagatgcccagatttcagaaggtgctagcagatcccttggtctccagttggccaaggtgaggagccaagagaacagctaccagagccgcctggatgacctcaagatgactgtggaaagagttcgggctctgggaagtcagtaccagaaccgagttcgggatactcacaggctcatcactcagatgcagctgagcctggcagaaagtgaagcttccttgggaaacactaacattcctgcctcagaccactacgtggggccaaatggctttaaaagtctggctcaggaggccacaagattagcagaaagccacgttgagtcagccagtaacatggagcaactgacaagggaaactgaggactattccaaacaagccctctcactggtgcgcaaggccctgcatgaaggagtcggaagcggaagcggtagcccggacggtgctgtggtgcaagggcttgtggaaaaattggagaaaaccaagtccctggcccagcagttgacaagggaggccactcaagcggaaattgaagcagataggtcttatcagcacagtctccgcctcctggattcagtgtctcggcttcagggagtcagtgatcagtcctttcaggtggaagaagcaaagaggatcaaacaaaaagcggattcactctcaagcctggtaaccaggcatatggatgagttcaagcgtacacagaagaatctgggaaactggaaagaagaagcacagcagctcttacagaatggaaaaagtgggagagagaaatcagatcagctgctttcccgtgccaatcttgctaaaagcagagcacaagaagcactgagtatgggcaatgccactttttatgaagttgagagcatccttaaaaacctcagagagtttgacctgcaggtggacaacagaaaagcagaagctgaagaagccatgaagagactctcctacatcagccagaaggtttcagatgccagtgacaagacccagcaagcagaaagagccctggggagcgctgctgctgatgcacagagggcaaagaatggggccggggaggccctggaaatctccagtgagattgaacaggagattgggagtctgaacttggaagccaatgtgacagcagatggagccttggccatggaaaagggactggcctctctgaagagtgagatgagggaagtggaaggagagctggaaaggaaggagctggagtttgacacgaatatggatgcagtacagatggtgattacagaagcccagaaggttgataccagagccaagaacgctggggttacaatccaagacacactcaacacattagacggcctcctgcatctgatgggtatgtgaacccacaacccacaaccttccagctccatgctccagggcttt

SEQ ID NO: 2 - protein, 1111 aa, Homo sapien [sequence omitted]

(19) United States
(12) Patent Application Publication
(10) Pub. No.: US 2010/0260764 A1
(43) Pub. Date: Oct. 14, 2010

(54) COMPOSITIONS AND METHODS FOR INHIBITING SQUAMOUS CELL CARCINOMA

(76) Inventor: M. Peter Marinkovich, Redwood City, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/733,418

(22) PCT Filed: Aug. 28, 2008
(86) PCT No.: PCT/US08/10291

§ 371 Date: May 20, 2010

Related U.S. Application Data
(60) Provisional application No. 60/966,827, filed on Aug. 29, 2007.

(51) Int. Cl. A61K 39/395; G01N 33/53

(52) U.S. Cl. 424/139.1; 424/172.1; 424/152.1; 435/7.1

(57) ABSTRACT

The present invention relates to compositions and methods for detecting and inhibiting squamous cell carcinoma using agents that target the laminin 332 γ2 processed region polypeptide.

### FEDERALLY SPONSORED RESEARCH AND DEVELOPMENT

This invention was made with Government support under contracts AR044012 and AR047223 awarded by the National Institutes of Health. The Government has certain rights in this invention.

### FIELD

The present invention relates to compositions and methods for detecting and inhibiting squamous cell carcinoma using agents that target the laminin-332 γ2 domain IV-V.

### INTRODUCTION

Squamous cell carcinoma (SCC) is common form of cancer and is the second most common form of skin cancer in the United States. SCCs are highly invasive and metastatic. SCCs are associated with a comparatively high risk of recurrence, resulting in significant mortality. SCC can be diagnosed by biopsy. However, SCCs are typically not as distinct as basal cell carcinomas or melanomas, making detection and diagnosis difficult. Current methods of treatment, i.e. surgery, radiotherapy, and chemotherapy, require continued monitoring due to the metastatic nature of the disease. The development of alternative methods of detection and treatment is therefore desirable.

The compositions and methods described herein are directed towards identifying agents that can detect and inhibit proteins associated with SCC tumorigenesis. Of particular interest, are agents that interact with the laminin-332 γ2 domain IV and/or domain V.

### SUMMARY

Provided herein are compositions and methods useful for detecting and treating squamous cell carcinoma (SCC). The compositions generally comprise antibodies capable of binding a γ2 processed region polypeptide. Polypeptides sequences of interest include SEQ ID NO:2, particularly SEQ ID NO:2, residues 1-434. The compositions can include additional components, such as, detectable labels and a pharmaceutically acceptable carrier.

The methods generally involve administering a therapeutically effective amount of a composition comprising one or more antibodies capable of inhibiting SCC tumorigenesis to a patient diagnosed with SCC. Treatment of a patient diagnosed with SCC with the compositions described herein can be combined with other medical means for treating SCC, such as surgery, radiotherapy, and chemotherapy. The SCC can be selected from the group consisting of skin cancer, lung cancer, head cancer, gastric cancer, colorectal cancer, throat cancer, cancer of the urinary tract, cancer of the reproductive tract, esophageal cancer, and bronchiogenic carcinoma.

Also provided herein are methods for screening for candidate agents that inhibit SCC tumorigenesis. In some embodiments, a method is provided for screening for candidate agents that inhibit SCC tumor development comprising the steps of: a) screening agent for specific binding to laminin-332 γ2 processed region polypeptide; b) subcutaneously injecting nude mice with a suspension comprising: i) Ras/IKB transformed epithelial cells; ii) a candidate agent found to bind specifically to laminin-332 γ2 processed region polypeptide; and b) determining the presence or absence of a tumor.

In some embodiments, a method is provided to evaluate the effect of a candidate SCC drug comprising administering the drug to a patient diagnosed with SCC and removing a cell sample from the patient. A number of different assays can be used to evaluate the effect of the candidate drug. For example, the expression profile of the cell sample can be determined and compared with an expression profile of a healthy individual. In some embodiments, the cell sample can be analyzed for the presence or absence of a γ2 processed region polypeptide before and after treatment with a candidate drug. In yet other embodiments, the size of the tumor before and after treatment with a candidate drug can be analyzed to determine if the drug is effective in inhibiting the invasion of nearby normal cells.

Also provided herein is a method for diagnosing SCC comprising removing a cell sample from an individual and analyzing the cell for the presence of γ2 processed region polypeptide.

These and other features of the compositions and methods described herein will become more apparent from the detailed description below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Aspects of the invention can be more fully understood with respect to the following drawings. In the drawings, similar elements are referenced with like numbers.

FIG. 1: 5 micron frozen sections of human skin or of two cuteneous squamous cell carcinoma tumors were analyzed by indirect immunofluorescent microscopy using antibodies against total laminin-332 (total lam), the laminin alpha 3 G45 domain (a3 G45) or the laminin gamma 2 domain IV-V. To the left, a schematic of laminin-332 and its major processing sites, marked by red arrows.

FIG. 2: Left: Results of analysis of 75 cases of paraffin embedded human cutaneous SCC using G45 pAb by immunohistochemical analysis. Upper left diagram shows number and percentage of samples which showed negative, moderate or strong expression. Scale bar=50 mm. Bottom: representative samples of 56 cases of paraffin embedded human extra-cutaneous SCC from various tissues using G45 pAb by immunohistochemical analysis showing moderate to strong expression. Scale bar=50 mm. Right: results of extra-cutaneous SCC tissue survey, showing tissues of origin, and intensity of staining for G45 pAb antibody.

FIG. 3: Left: Results of analysis of 56 cases of paraffin embedded human SCC of various tissue origins using anti-laminin gamma 2 domain IV-V pAb by immunohistochemical analysis. Right: representative samples of 56 cases of paraffin embedded human extra-cutaenous SCC from various tissues using anti-laminin gamma 2 domain IV-V pAb by immunohistochemical analysis showing moderate to strong expression.

FIG. 4: Laminin gamma 2 null human keratinocytes obtained from patients with junctional epidermolysis bullosa were retrovirally transduced with cDNA coding for LAcZ (control) full length human gamma 2 chain (full length g2), or human laminin gamma 2 chain containing a deletion of amino acids 1-434 (truncated g2). Cells were then transformed with Ras/IKBa cDNA transfer and resulting SCC tumors were measured at 3 and 4 weeks after subcutaneous injection to immunodeficient mice.

FIG. 5. LAMININ γ2domain IV-V is highly expressed in SCC but not in normal tissues. A tumor survey (left) and IDIF (bottom) is shown using a pAb antibody to γ2domain IV-V, shown in red in the schematic to the right. A pAb to total laminin-332 (total lam) is shown for comparison.

FIG. 6. Laminin γ2 chain is required for human SCC. Top, truncation constructs of γ2 chain. Bottom, tumor growth after 5 weeks with transformed cells containing either deletions of or antibodies to the γ2 chain domain IV-V.

### DETAILED DESCRIPTION OF THE EMBODIMENTS

It is to be understood that both the foregoing general description and the following detailed description are exemplary and explanatory only and are not restrictive of the inventions described herein. In this application, the use of the singular includes the plural unless specifically state otherwise. Also, the use of “or” means “and/or” unless state otherwise. Similarly, “comprise,” “comprises,” “comprising,” “include,” “includes” and “including” are not intended to be limiting. Unless defined otherwise, all technical and scientific terms used herein have the same meanings as commonly understood by one of ordinary skill in the art to which the invention belongs.

### 1. DEFINITIONS

As used herein, the following terms and phrases are intended to have the following meanings:

“Antibody” has its standard meaning and is intended to refer to intact molecules as to fragments thereof, such as Fab, F(ab′)₂, and Fv fragments, that are capable of binding an epitope.

“Cancer” has its standard meaning and is intended to refer to any malignant tumor of potentially unlimited growth that expands locally by proliferation and systemically by metastasis.

“Neoplasm” has its standard meaning and is intended to refer to the abnormal growth of a tissue, such as a tumor.

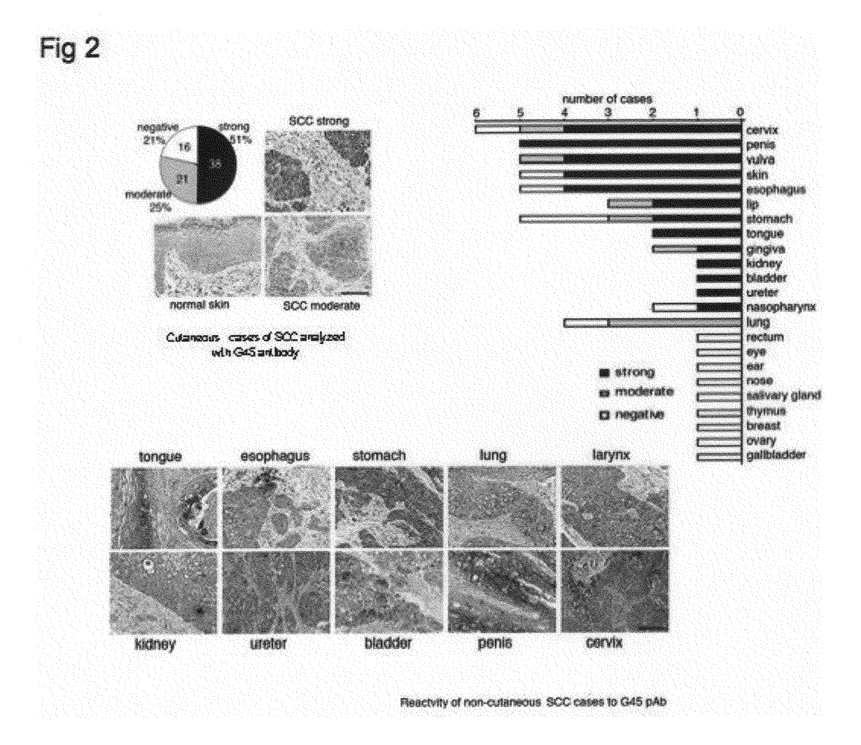

“Nucleobase” means those naturally occurring and those synthetic nitrogenous, aromatic moieties commonly found in the nucleic acid arts. Examples of nucleobases include purines and pyrimidines, genetically encoding nucleobases, analogs of genetically encoding nucleobases, and purely synthetic nucleobases. Specific examples of genetically encoding nucleobases include adenine, cytosine, guanine, thymine, and uracil. Specific examples of analogs of genetically encoding nucleobases and synthetic nucleobases include 5-methylcytosine, pseudoisocytosine, 2-thiouracil and 2-thiothymine, 2-aminopurine, N9-(2-amino-6-chloropurine-), N9-(2,6-diaminopurine), hypoxanthine, N9-(7-deaza-guanine), N9-(7-deaza-8-aza-guanine) and N8-(7-deaza-8-aza-adenine). 5-propynyl-uracil, 2-thio-5-propynyl-uracil. Other non-limiting examples of suitable nucleobases include those nucleobases illustrated in FIGS. 2(A) and 2(B) of U.S. Pat. No. 6,357,163, incorporated herein by reference in its entirety.

“Nucleoside” refers to a nucleobase linked to a pentose sugar. Pentose sugars include ribose, 2′-deoxyribose, 3′-deoxyribose, and 2′,3′-dideoxyribose.

“Nucleoside analog” refers to a nucleobase linked to a sugar, other than a pentose sugar. For example, a nucleobase linked to hexose.

“Nucleotide” refers to compound comprising a nucleobase, a pentose sugar and a phosphate. Thus, as used herein a nucleotide refers to a phosphate ester of a nucleoside, e.g., a triphosphate.

“Nucleobase Polymer or Oligomer” refers to two or more nucleobases that are connected by linkages that permit the resultant nucleobase polymer or oligomer to hybridize to a polynucleotide having at least a partially complementary nucleobase sequence. Nucleobase polymers or oligomers include, but are not limited to, poly- and oligonucleotides (e.g., DNA and RNA polymers and oligomers), poly- and oligonucleotide analogs and poly- and oligonucleotide mimics, such as polyamide nucleic acids or peptide nucleic acids. Polyamide nucleic acids and peptide nucleic acids are two different phrases used in the literature to describe the same molecule, abbreviated herein as PNA. Nucleobase polymers or oligomers can vary in size from a few nucleobases, for example, from 2 to 40 nucleobases, to several hundred nucleobases, to several thousand nucleobases, or more.

“Polynucleotides or Oligonucleotides” refer to nucleobase polymers or oligomers in which the nucleobases are linked by sugar phosphate linkages (sugar-phosphate backbone). Exemplary poly- and oligonucleotides include polymers of 2′-deoxyribonucleotides (DNA) and polymers of ribonucleotides (RNA). A polynucleotide may be composed entirely of ribonucleotides, entirely of 2′-deoxyribonucleotides or combinations thereof.

“Polynucleotide or Oligonucleotide Analog” refers to nucleobase polymers or oligomers in which the nucleobases are linked by a phosphate backbone comprising one or more sugar analogs or phosphate analogs. Typical oligonucleotide or polynucleotide analogs include, but are not limited to, sugar alkylphosphonates, sugar phosphoramidites, sugar alkyl- or substituted alkylphosphotriesters, sugar phosphorothioates, sugar phosphorodithioates, sugar phosphates and sugar phosphate analogs in which the sugar is other than 2′-deoxyribose or ribose, nucleobase polymers having positively charged sugar-guanidyl interlinkages such as those described in U.S. Pat. No. 6,013,785 and U.S. Pat. No. 5,696,253 (see also, Dagani 1995, Chem. & Eng. News 4-5:1153; Dempey et al., 1995, J. Am. Chem. Soc. 117:6140-6141). Such positively charged analogues in which the sugar is 2′-deoxyribose are referred to as “DNGs,” whereas those in which the sugar is ribose are referred to as “RNGs.” Specifically included within the definition of poly- and oligonucleotide analogs are locked nucleic acids (LNAs; see, e.g. Elayadi et al., 2002, Biochemistry 41:9973-9981; Koshkin et al., 1998, J. Am. Chem. Soc. 120:13252-3; Koshkin et al., 1998, Tetrahedron Letters, 39:4381-4384; Jumar et al., 1998, Bioorganic & Medicinal Chemistry Letters 8:2219-2222; Singh and Wengel, 1998, Chem. Commun., 12:1247-1248; WO 00/56746; WO 02/28875; and, WO 01/48190; all of which are incorporated herein by reference in their entireties).

“Polynucleotide or oligonucleotide mimic” refers to nucleobase polymers or oligomers in which the nucleobases are connected by a linkage other than a sugar-phosphate linkage or a sugar-phosphate analog linkage. Mimics with a specific linkage include peptide nucleic acids (PNAs) as described in any one or more of U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,451,968, 6,441,130, 6,414,112 and 6,403,763; all of which are incorporated herein by reference. Other types of mimics are described in the following publications: Lagriffoul et al., 1994, Bioorganic & Medicinal Chemistry Letters, 4: 1081-1082; Petersen et al., 1996, Bioorganic & Medicinal Chemistry Letters, 6: 793-796; Diderichsen et al, 1996, Tett. Lett. 37: 475-478; Fujii et al., 1997, Bioorg. Med. Chem. Lett. 7: 637-627; Jordan et al., 1997, Bioorg. Med. Chem. Lett. 7: 687-690; Krotz et al., 1995, Tett. Lett. 36: 6941-6944; Lagriffoul et al, 1994, Bioorg. Med. Chem. Lett. 4: 1081-1082; Diederichsen, U., 1997, Bioorganic & Medicinal Chemistry 25 Letters, 7: 1743-1746; Lowe et al., 1997, J. Chem. Soc. Perkin Trans. 1, 1: 539-546; Lowe et al., 1997, J. Chem. Soc. Perkin Trans. 11: 547-554; Lowe et al., 1997, I. Chem. Soc. Perkin Trans. 1 1:5 55-560; Howarth et al., 1997, I. Org. Chem. 62: 5441-5450; Altmann, K-H et al., 1997, Bioorganic & Medicinal Chemistry Letters, 7: 1119-1122; Diederichsen, U., 1998, Bioorganic & Med. Chem. Lett., 8:165-168; Diederichsen et al., 1998, Angew. Chem. mt. Ed., 37: 302-305; Cantin et al., 1997, Tett. Lett., 38: 4211-4214; Ciapetti et al., 1997, Tetrahedron, 53: 1167-1176; Lagriffoule et al., 1997, Chem. Eur. 1.′ 3: 912-919; Kumar et al., 2001, Organic Letters 3(9): 1269-1272; and the Peptide-Based Nucleic Acid Mimics (PENAMs) of Shah et al. as disclosed in WO 96/04000. All of which are incorporated herein by reference.

The oligonucleotides may be single stranded or double stranded, as specified, or contain portions of both double stranded or single stranded sequence. As will be appreciated by those in the art, the depiction of a single strand (“Watson”) also defines the sequence of the other strand (“Crick”); thus the sequences described herein also includes the complement of the sequence.

“Protein” has its standard meaning and is intended to refer to at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids [see Simon et al., Proc. Natl. Acad. Sci. U.S.A. 89(20:9367-71 (1992)], generally depending on the method of synthesis. Thus “amino acid”, or “peptide residue”, as used herein means both naturally occurring and synthetic amino acids. For example, homo-phenylalanine, citrulline, and noreleucine are considered amino acids for the purposes of the invention. “Amino acid” also includes imino acid residues such as proline and hydroxyproline. In addition, any amino acid representing a component of the variant proteins of the present invention can be replaced by the same amino acid but of the opposite chirality. Thus, any amino acid naturally occurring in the L-configuration (which may also be referred to as the R or S, depending upon the structure of the chemical entity) may be replaced with an amino acid of the same chemical structural type, but of the opposite chirality, generally referred to as the D-amino acid but which can additionally be referred to as the R— or the S—, depending upon its composition and chemical configuration. Such derivatives generally have the property of greatly increased stability, and therefore are advantageous in the formulation of compounds which may have longer in vivo half lives, when administered by oral, intravenous, intramuscular, intraperitoneal, topical, rectal, intraocular, or other routes.

In some embodiments, the amino acids are in the (S) or L-configuration. If non-naturally occurring side chains are used, non-amino acid substituents may be used, for example to prevent or retard in vivo degradations. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. S218: U138 Part 2 Aug. 22, 1999, both of which are expressly incorporated by reference herein.

“Squamous cell carcinoma” has its standard meaning and is intended to refer to any neoplasm or tumor of epithelial cells.

“Tumorigenesis” has its standard meaning and is intended to refer to the basic developmental processes that produce tumors. These basic properties include the ability to proliferate or invade nearby normal cells and the ability to migrate from the site where the tumor initiated, i.e. metastasis.

### Exemplary Embodiments

Provided herein are: (1) laminin-332 γ2 domain IV and/or domain V polypeptides; (2) antibodies which specifically bind to such polypeptide, thereby inhibiting tumorigenesis of neoplastic epithelial cells; (3) methods for screening for agents, such as antibodies, small molecules, etc., that specifically bind one or more of the γ2 polypeptides described herein; (4) methods for screening for agents that inhibit squamous cell carcinoma (SCC) tumor development using one or more of the γ2 polypeptides, (5) methods for diagnosing SCC; and, (6) methods for determining the efficacy of candidate agents used to treat SCC. All of these inventions rely upon laminin-332 γ2 domain IV and/or domain V polypeptides, nucleic acids that encode laminin-332 γ2 domain IV and/or domain V polypeptides and other molecules, such as antibodies, that bind laminin-332 γ2 domain IV and/or domain V polypeptides.

Laminin-332 (formerly called laminin-5; kalinin, nicein, or BM6000) is a heterotrimeric extracellular matrix protein that is initially synthesized and secreted in an unprocessed form with an α3 chain of 200 kDa, a β3 chain of 140 kDA, and a γ2 chain of 155 kDA. (Marinkovich et al., 1992, J. Biol. Chem., 267: 17900-17906). Laminin-5 is a component of the basal lamina, the structure that provides tissue integrity, as well as the foundation for migration, growth and differentiation of cells.

Large deposits of laminin-5 are found at the leading edges of squamous cell carcinomas (SCCs). This deposition of laminin-5 is believed to serve as a substrate for tumor invasion (see, e.g., Pyke et al., 1995, Canc. Res. 55: 4132-4139; Berndt et al., 1997, Invasion and Metastasis, 17: 251-258). Increased laminin-5 immunoreactivity is indicative of a poor prognosis in patients with squamous cell carcinoma (SCC). Laminin-5 is also preferentially expressed by invading malignant cells of many human carcinomas in additions to SCCs, colon and mammary carcinomas (Pyke, et al., 1994, Am. J. Pathol. 145(4):782-791) and malignant gliomas (Fukushima et al., 1998, Int. J. Cancer, 76: 63-72).

Processing of extracellular matrix proteins by proteases is emerging as a key mechanisms in processes such as wound healing and tumor metastasis. Several proteases have been implicated in laminin-5-processing (see, e.g., Veitch et al., 2003, J. Biol. Chem., 278: 15661-15668; and U.S. patent Pub. No. 2002/0076736). In fully formed tissues, laminin-5 is completely processed and is devoid of the G4 and G5 domains (Marinkovich et al., 1992, J. Biol. Chem., 267:17900-17906). Without being bound by theory, it appears that specific proteolytic processing can convert laminin-5 from a pro-migratory signal required for cell migration during tumor invasion and tissue remodeling to an adhesive substrate devoid of the G4 and G5 domains. The processing of laminin γ2 is described, for example, by Amano, et al. (2000) J Biol Chem 275, 22728-22735, herein specifically incorporated by reference for teachings of laminin processing sites.

“SCC” refers herein to any malignant neoplasm or tumor of epithelial cells. Specific examples of epithelial cells include squamous cells, squamous carcinoma cells, keratinocytes, mucosal epithelial cells, such as oral mucosal cells, gastrointestinal epithelial cells, corneal epithelium of the eye, and epithelial cells of the urinary and reproductive tract. Specific examples of SCC carcinomas arising from neoplastic epithelial cells include skin, lung, head, neck, oral, gastric, colorectal, throat, urinary tract, reproductive tract, esophageal, etc.

SCC is commonly sun-induced, i.e., actinically derived SCC. SCC can also result from transplant or invasive surgery, or follow other immunosuppressive situations. Chronic inflammation can lead to development of SCC at the site of inflammation, e.g., a burn or scar, Majolin's ulcer, etc. SCC can be virally induced, for example, SCC can result from human papillomavirus-induced (HPV) infection. SCC can include adenoid (acantholytic) SCC, spindle cell SCC, verrucous carcinoma (VC), keratoacanthoma (KA), nodular SCC periungual SCC, and other epithelial carcinomas.

Sequences of interest relate to the laminin-332 γ2 domain IV and/or domain V polypeptides (the sequence of γ2 chain is provided herewith as SEQ ID NO:1, SEQ ID NO:2). Polypeptides of various lengths spanning domain IV and/or domain V can be generated. For example, a polynucleotide spanning the processed region of the human γ2 chain of laminin 332 can be generated by starting at nucleotide position 1 and ending at nucleotide 1302. Within the processed region (with reference to SEQ ID NO:2) are multiple laminin-type epidermal growth factor-like domains, at amino acids 83-127 and 139-185, and a laminin B domain at amino acids 245-370. The nucleotide sequences for these domains correspond to SEQ ID NO:1, nucleotides 249-381 and 417-555 for the EGF-like domains, and 735-1110 for the laminin B domain.

The exact number of nucleotides or nucleotide analogs chosen will vary depending on the sequence of the nucleotides selected and the presence of nucleotides encoding amino acids that comprise antigenic determinants. By “epitope” or “determinant” “or antigenic determinant” herein is meant a portion of a protein that can generate and/or bind an antibody; or T-cell receptor in the context of MHC. For example, the presence of antigenic determinants within the domains can be identified by searching databases for MHC ligands and peptide motifs (Rammensee, H., et al. (1999) Immunogenetics, 50:213-219). This information can be used to generate MHC epitopes. Typically, epitopes recognized by MHC class I molecules comprise between 8 and 11 amino acids, thus, an genetic sequence encoding an MHC class I epitope can range between 24 to 33 nucleotides. Viral peptides recognized by MHC class II molecules comprise between 10 to 20 amino acids, thus, a genetic sequence encoding an MHC class II epitope can range between 30 to 60 nucleotides (Fundamental Immunology, 4th edition, W. E. Paul, ed., Lippincott-Raven Publishers, 1999, Chapter 39, pp 1295-1334). In other embodiments, genetic sequences range between 24 to 1302, or from 60 to 300 nucleotides, or from 60 to 405 nucleotides, or from 60 to 555 nucleotides, or from 60 to 600 nucleotides, or from 60 to 750 nucleotides, or from 60 to 900 nucleotides or from 60 to 1050 nucleotides. In yet other embodiments, coding sequences range from 150 to 300 nucleotides, or from 150 to 405 nucleotides, or from 150 to 450 nucleotides, or from 150 to 525 nucleotides, or from 150 to 600 nucleotides, or from 150 to 750 nucleotides, or from 150 to 1050 nucleotides, or from 300 to 600 nucleotides, or from 300 to 900 nucleotides, or from 300 to 1050 nucleotides.

In some embodiments, polypeptides are generated from the amino acid sequence encoding the laminin-332 γ2 domain IV and/or domain V polypeptides or subdomains thereof. Within the processed region (with reference to SEQ ID NO:2) are multiple laminin-type epidermal growth factor-like domains, at amino acids 83-127 and 139-185, and a laminin B domain at amino acids 245-370. Such polypeptides may be recombinant. A “recombinant protein” is a protein made using recombinant techniques, i.e. through the expression of a recombinant oligonucleotide as described above. A recombinant protein is distinguished from a naturally occurring protein by at least one or more characteristics. For example, the protein may be isolated or purified away from some or all of the proteins and compounds with which it is normally associated in its wild type host, and thus may be substantially pure. Generally, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about. 90% being particularly preferred. The definition includes the production of an polypeptide from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

Polypeptides of various lengths spanning the γ2 processed region can be generated. The exact number of amino acids or amino acid analogs chosen will vary depending on the sequence of the amino acids selected, the presence of cleavage sites, and the presence of amino acids comprising antigenic determinants.

As discussed above, the presence of antigenic determinants within the γ2 processed region can be identified by searching databases for MHC ligands and peptide motifs (Rammensee, H., et al. (1999) Immunogenetics, 50:213-219). This information can be used to identify MHC epitopes. Typically, epitopes recognized by MHC class I molecules comprise between 8 and 11 amino acids while epitopes recognized by MHC class II molecules comprise between 10 to 20 amino acids (Fundamental Immunology, 4th edition, W. E. Paul, ed., Lippincott-Raven Publishers, 1999, Chapter 39, pp 1295-1334). Thus, in some embodiments, sequences of interest range between 8 to 11. In other embodiments, sequences of interest range between 10 to 20 amino acids. In other embodiments, sequences of interest range from 8 to 350 amino acids. In still other embodiments, sequences of interest range between 20 to 100 amino acids, or from 20 to 135 amino acids, or from 20 to 185 amino acids, or from 20 to 200 amino acids, or from 20 to 250 amino acids, or from 20 to 300 amino acids or from 20 to 350 amino acids. In yet other embodiments, sequences of interest range from 50 to 100 amino acids, or from 50 to 135 amino acids, or from 50 to 150 amino acids, or from 50 to 175 amino acids, or from 50 to 200 amino acids, or from 50 to 250 amino acids, or from 50 to 350 amino acids, or from 100 to 200 amino acids, or from 100 to 300 amino acids, or from 100 to 350 amino acids.

The sequences of interest may be unprocessed or processed. As used herein “unprocessed” refers to a sequence of interest that is still associated with the laminin 332 γ2 chain. By “processed” herein is meant that the peptide is dissociated SEQ ID NO:2 residues 1-434.

### Expression Systems

Polynucleotides encoding sequences of interest can be used to make a variety of expression vectors to express sequences of interest which can then be used in the diagnostic, screening and therapeutic applications described below. The expression vectors may be either self-replicating extrachromosomal vectors or vectors which integrate into a host genome. Generally, these expression vectors include transcriptional and translational regulatory nucleic acid operably linked to the oligonucleotide encoding the protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

An oligonucleotide is “operably linked” when it is placed into a functional relationship with another olignonucleotide sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, synthetic oligonucleotide adaptors or linkers are used in accordance with conventional practice. The transcriptional and translational regulatory nucleic acid will generally be appropriate to the host cell used to express the protein; for example, transcriptional and translational regulatory nucleic acid sequences from Bacillus are preferably used to express the protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

Laminin 332 γ2 processed region polypeptides are produced by culturing a host cell transformed with an expression vector containing an oligonucleotide encoding a γ2 processed region polypeptide, under the appropriate conditions to induce or cause expression of the γ2 processed region polypeptide. The conditions appropriate for γ2 processed region polypeptide expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect, plant and animal cells, including mammalian cells. Of particular interest are primary human keratinocytes, although other cells also can be used, i.e. Drosophila melanogaster cells, Saccharomyces cerevisiae and other yeasts, E. coli, Bacillus subtilis, 293 cells, CHO, other human cell and cell lines.

In some embodiments, the laminin 332 γ2 domain peptides are expressed in mammalian cells. Mammalian expression systems are also known in the art, and include retroviral systems. A preferred expression vector system is a retroviral vector system such as is generally described in Dajee et al., 2003, Nature, 421: 639-643, which is incorporated herein by reference in its entirety. Of particular use as mammalian promoters are the promoters from mammalian viral genes, since the viral genes are often highly expressed and have a broad host range. Examples include, but are not limited to, the SV40 early promoter, mouse mammary tumor virus LTR promoter, adenovirus major late promoter, herpes simplex virus promoter, and the CMV promoter. Typically, transcription termination and polyadenylation sequences recognized by mammalian cells are regulatory regions located 3′ to the translation stop codon and thus, together with the promoter elements, flank the coding sequence. Examples of transcription terminator and polyadenlytion signals include those derived form SV40.

For example, the full length laminin 332 γ2 cDNA can be ligated into a pENTR1A™ vector (Invitrogen). The full length laminin 332 γ2 cDNA can be cleaved and the PCR used to obtain an oligonucleotide sequence from domain IV and/or domain V. The resulting PCR product can be ligated into a vector and the cloning product confirmed by sequencing. The cloned product can then be transferred from the vector to a Gateway adapted LSRZ retroviral vector through lambda phage recombination. See Dajee et al., 2003, Nature, 421: 639-643.

In some embodiments, laminin 332 γ2 domain peptides are expressed in bacterial systems. Bacterial expression systems are well known in the art. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the laminin 332 γ2 domain peptide in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

Various oligonucleotides can be generated from the laminin 332 γ2 domain IV and/or domain V coding sequences and subcloned into a retroviral vector. The resulting retroviral vectors can be transduced into cell cultures and the cells analyzed for cell scattering and cell migration. Alternatively, laminin-332 negative primary human keratinocytes co-expressing Ras, a stable NE-κB repressor mutant of IκBα (i.e. IKB), and one or more laminin 332 γ2 domain peptide(s) can be retrovirally transduced and used to regenerate human skin on immune deficient mice (i.e. nude mice). The subsequent development of neoplasms can be monitored and compared to wild type mice.

In some embodiments, matrigel, which contains heparin sulfate proteoglycan, is used as a matrix for the suspension of RAS/IKB transformed keratinocytes prior to subcutaneous injection into nude mice. In other embodiments, laminin 332 γ2 domain peptides can be suspended in matrigel prior to injection of RAS/IKB transformed keratinocytes.

In some embodiments, γ2 processed region polypeptide are purified or isolated after expression. Polypeptides may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the γ2 processed region polypeptide may be purified using a standard antibody column. Ultrafiltration and diafiltration techniques, in conjunction with protein concentration, are also useful. For general guidance in suitable purification techniques, see Scopes, R., Protein Purification, Springer-Verlag, N.Y. (1982). The degree of purification necessary will vary depending on the use of the γ2 processed region polypeptide protein. In some instances no purification will be necessary.

### Antisense Sequences

The coding sequences can be used in a variety of different ways. In some embodiments, antisense therapeutic agents affect the expression and activity of laminin 332 γ2 domain peptides. Antisense technology relies on the modulation of expression of a target protein through the specific binding of an antisense sequence to a target sequence encoding the target protein or directing its expression. (See, e.g., Agrawal, S., ed., 1996, Antisense Therapeutics, Humana Press Inc., Totawa N.J.; Alama et al. (1997) Pharmacol Res. 36(3):171-178; Crooke, S. T., 1997, Adv. Pharmacol. 40:1-49; and Lavrosky et al., 1997, Biochem. Mol. Med. 62(1):11-22.). Antisense sequences are nucleic acid sequences capable of specifically hybridizing to at least a portion of a target sequence. Antisense sequences can bind to cellular mRNA or genomic DNA, blocking translation or transcription and thus interfering with expression of a targeted protein product. Antisense sequences can be any nucleic acid material, including DNA, RNA, or any nucleic acid mimics or analogs. (See, e.g., Rossi et al., 1991, Antisense Res. Dev. 1(3):285-288; Pardridge et al., 1995, Proc. Nat. Acad. Sci. 92 (12):5592-5596; Nielsen, P. E. and G. Haaima, 1997, Chem. Soc. Rev. 96:73-78; and Lee et al., 1998, Biochemistry 37 (3):900-1010.). Delivery of antisense sequences can be accomplished in a variety of ways, such as through intracellular delivery using an expression vector. Site-specific delivery of exogenous genes is also contemplated, such as techniques in which cells are first transfected in culture and stable transfectants are subsequently delivered to the target site.

Typically, antisense oligonucleotides between 15 to 25 nucleobases or nucleobase analogs are capable of producing the desired therapeutic effect, i.e., direct disruption of translation of laminin 332. In addition, chemically reactive groups, such as iron-linked ethylenediamine-tetraacetic acid (EDTA-Fe), can be attached to antisense oligonucleotides, causing cleavage of the RNA at the site of hybridization. These and other uses of antisense methods to inhibit the in vitro translation of genes are well known in the art (see, e.g., Marcus-Sakura (1988) Anal. Biochem. 172:289).

In some embodiments, antisense oligonucleotides are designed such that they disrupt the translation of the laminin 332 γ2 chain. Delivery of antisense agents can be achieved intracellularly through using a recombinant expression vector such as a chimeric virus or a colloidal dispersion system which, upon transcription, produces a sequence complementary to at least a portion of the cellular sequence encoding the target protein (see, e.g., Slater et al., 1998, J. Allergy Cli. Immunol. 102 (3): 469-475). Delivery of antisense sequences can also be achieved through various viral vectors, including retrovirus and adeno-associated virus vectors. (See, e.g., Miller, 1990, Blood, 76: 271; and Uckert and Walther, 1994, Pharacol. Ther., 63(3): 323-347). Suitable viral vectors include, but are not limited to, adenoviruses, herpes viruses, vaccinia, and RNA viruses such as retroviruses.

Retroviral vectors can be derivatives of murine or avian retrovirus. Retroviral vectors can be made target-specific by inserting, for example, a polynucleotide encoding a protein or proteins such that the desired ligand is expressed on the surface of the viral vector. The ligand can be a glycolipid carbohydrate or protein. Preferred targeting can also be accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector containing the antisense polynucleotide. See, e.g, WO 91/04753.

Other delivery mechanisms that can be used for delivery of antisense sequences to target cells include colloidal dispersion and liposome-derived systems, artificial viral envelopes, and other systems available to one of skill in the art (see, e.g., Rossi, 1995, Br. Med. Bull. 51 (1): 217-225; Morris et al., 1997, Nucl. Acids Res. 25 (14): 2730-2736; Boado et al., 1998, J. Pharm. Sci. 87 (11): 1308-1315; and WO 90/10448). For example, delivery systems can make use of macromolecule complexes, nanocapsules, microspheres, beads, and lipid-based systems including oil-in-water emulsions, micelles, mixed micelles, and liposomes.

### Antibodies

In some embodiments, the laminin 332 γ2 processed region polypeptide are used to generate antibodies that can be used in the screening and therapeutic applications described herein. Preferably, the polypeptide should comprise at least one epitope or determinant. In some embodiments, the epitope is unique; that is, antibodies generated to a unique epitope show little or no cross-reactivity. The term “antibody” can include polyclohal antibodies, monoclonal antibodies, chimeric antibodies, antibody fragments, such as Fab, Fab₂, single chain antibodies (Fv for example) etc., either produced by the modification of whole antibodies or those synthesized de novo using recombinant DNA technologies.

Laminin γ2 polypeptides can be evaluated to determine regions of immunogenicity. As discussed above, methods of analysis and epitope selection are well-known in the art. Analysis and selection can also be accomplished, for example, by various software packages, such as LASERGENE NAVIGATOR software (DNASTAR; Madison, Wis.). The polypeptides or fragments used to induce antibodies should be antigenic, but need not necessarily be biologically active. An antigenic fragment or polypeptide is at least 5 amino acids in length, more preferably, at least 10 amino acids in length, and most preferably, at least 15 amino acids in length. It is preferable that the antibody-inducing fragment or polypeptide is identical to at least a portion of the amino acid sequence of the γ2 processed region polypeptide, or subdomains thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor, and antibodies can be produced against the chimeric molecule.

Methods for the production of antibodies are well-known in the art. For example, various hosts, including goats, rabbits, rats, mice, humans, and others, may be immunized by injection with the polypeptide of the invention or any immunogenic fragment or peptide thereof. Depending on the host species, various adjuvants may be used to increase immunological response. Such adjuvants include, but are not limited to, Freund's adjuvant, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

Monoclonal and polycolonal antibodies can be prepared using any technique which provides for the production of antibody molecules by continuous cell lines in culture. Techniques for in vivo and in vitro production are well-known in the art (see, e.g., Pound, J. D., 1998, Immunochemical Protocols, Humana Press, Totowa N.J.; Harlow, E. and D. Lane, 1988, Antibodies, A Laboratory Manual, Cold Spring Harbor Laboratory, New York). The production of chimeric antibodies is also well-known, as is the production of single-chain antibodies (see, e.g., Morrison et al., 1984, Proc. Natl. Acad. Sci. 81: 6851-6855; Neuberger et al., 1984, Nature, 312: 604-608; Takeda et al., 1985, Nature, 314: 452-454). Antibodies with related specificity, but of distinct idiotypic composition, may be generated, for example, by chain shuffling from random combinatorial immunoglobin libraries (see, e.g., Burton, 1991, Proc. Natl. Acad. Sci. 88: 11120-11123).

Antibodies may also be produced by inducing in vivo production in the lymphocyte population or by screening immunoglobulin libraries or panels of highly specific binding reagents (see, e.g., Orlandi et al., 1989, Proc. Natl. Acad. Sci. 86: 3833-3837; Winter, G. and C. Milstein, 1991, Nature, 349: 293-299). Antibody fragments which contain specific binding sites for the target polypeptide may also be generated. Such antibody fragments include, but are not limited to, F(ab′)₂ fragments, which can be produced by pepsin digestion of the antibody molecule, and Fab fragments, which can be generated by reducing the disulfide bridges of the F(ab′)₂ fragments. Alternatively, Fab expression libraries may be constructed to allow rapid and easy identification of monoclonal Fab fragments with the desired specificity (see, e.g., Huse et al., 1989, Science, 254: 1275-1281).

In some embodiments, the antibodies are bispecific antibodies. Bispecific antibodies are monoclonal, preferably human or humanized, antibodies that have binding specificities for at least two different antigens. In the present case, one of the binding specificities is for a human laminin 5 alpha 3 G4-5 domain, and the other one is for any other antigen, such as a cell-surface'protein or receptor or receptor subunit, preferably one that is tumor specific.

In some embodiments, the antibodies are humanized antibodies. Humanized forms of non-human (e.g., murine) antibodies are chimeric molecules of immunoglobulins, immunoglobulin chains or fragments thereof (such as Fv, Fab, Fab′, F(ab′)₂ or other antigen binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework residues (FR) regions are those of a human immunoglobulin consensus sequence. The humanized antibody optimally also will comprise at least a portion of an immunoglobulin constant region (Fc), typically that of a human immunoglobulin (Jones et al., 1986, Nature, 321: 522-525; Riechmann et al., 1988, Nature, 332: 323-329; and Presta, 1992, Curr. Op. Struct. Biol., 2: 593-596).

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (Jones et al., 1986, Nature, 321: 522-525; Riechmann et al., 1988, Nature, 332: 323-329; Verhoeyen et al., 1988, Science, 239: 1534-1536), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, 1991, J. Mol. Biol., 227: 381; Marks et al., 1991, J. Mol. Biol., 222: 581). The techniques of Cole et al. and Boerner et al. are also available for the preparation of human monoclonal antibodies (Cole et al., 1985, Monoclonal Antibodies and Cancer Therapy, Alan R. Liss, p. 77, and Boerner et al., 1991, J. Immunol., 147(1): 86-95). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., 1992, Bio/Technology, 10: 779-783; Lonberg et al., 1994, Nature, 368: 856-859; Morrison, 1994, Nature, 368: 812-13; Fishwild et al., 1996, Nature Biotechnology, 14: 845-51; Neuberger, 1996, Nature Biotechnology, 14: 826; Lonberg and Huszar, 1995, Intern. Rev. Immunol. 13 65-93.

Antibodies can be tested for activity using a variety of methods well-known in the art. Various techniques may be used for screening to identify antibodies having the desired specificity, including various immunoassays, such as enzyme-linked immunosorbent assays (ELISAs), including direct and ligand-captyre ELISAs, radioimmunoassays (RIAs), immunoblotting, and fluorescent activated cell sorting (FACS). Numerous protocols for competitive binding or immunoradiometric assays, using either polyclonal or monoclonal antibodies with established specificities, are well known in the art (see, e.g., Harlow and Lane, supra). Such immunoassays typically involve the measurement of complex formation between the target polypeptide and a specific antibody. A two-site, monoclonal-based immunoassay utilizing monoclonal antibodies reactive to two non-interfering epitopes on the target polypeptide is preferred, but other assays, such as a competitive binding assay, may also be employed (see, e.g. Maddox et al., 1983, J Exp Med, 158: 1211).

Once made, the antibodies can be used to identify laminin 332 γ2 domain peptides in a sample, e.g., from biopsied tissue, etc. The amount of protein or mRNA can be determined using methods well known in the art, including but not limited to, quantitative image analysis, and reverse transcriptase polymerase chain reaction (RT-PCR) using portions of the biopsied tissue. Quantitation of mRNA can be determined by a competition reaction using equal volumes of the patient sample run against a series of decreasing known concentrations, e.g., of a mimic or mutant cDNA fragment.

Antibodies of interest are capable of specifically binding to laminin 332 γ2 domain peptides. By “specifically binding” herein is meant that the antibodies bind to the protein with a binding constant in the range of at least 10⁻⁴-10⁻⁹ M⁻¹, preferably in the range of 10⁻⁷-10⁻⁹ M⁻¹.

In some embodiments, antibodies are capable of reducing or eliminating the biological activity or function of the laminin 332 γ2 domain peptide(s). That is, the addition antibodies (i.e., polyclonal or monoclonal) to SCC or neoplastic epithelial cells expressing a laminin 332 γ2 domain IV or domain V peptide reduces or eliminates the activity. Generally, at least a 25% decrease in activity is preferred, with at least about 50% being particularly preferred and about a 95-100% decrease being especially preferred.

In some embodiments, antibodies are conjugated to a therapeutic moiety. For example, the therapeutic moiety can be an agent that inhibits enzymatic activity such as protease or protein kinase activity associated with SCC. In other embodiments, the therapeutic moiety can be a cytotoxic agent. Cytotoxic agents are numerous and varied and include, but are not limited to, radiochemicals, cytotoxic drugs or toxins or active fragments of such toxins. Suitable toxins and their corresponding fragments include diphtheria A chain, exotoxin A chain, ricin A chain, abrin A chain, curcin, crotin, phenomycin, enomycin and the like.

### Diagnosis and Therapy

The laminin 332 γ2 processed region polypeptide and coding sequences can be used in a variety of different ways. For example, the peptides and coding sequences can be used in diagnostic assays, screening assays, and in therapeutic application. In some embodiments, the peptides and antibodies to the peptides are used diagnostic markers for the detection of SCC. Detection of laminin 332 γ2 processed region polypeptideS in putative SCC tissue or patients allows for a determination or diagnosis of SCC. To detect or diagnose SCC, baseline values for the expression or activity are established in order to provide a basis for the diagnosis and/or prognosis of SCC in a patient. In some embodiments, this is accomplished by combining body fluids, tissue biopsies, or cell extracts taken from normal subjects with one or more antibody(ies) to a laminin 332 γ2 processed region polypeptide under conditions suitable for complex formation. Such conditions are well known in the art. The amount of standard complex formation may be quantified by comparing levels of antibody-target complex in the normal sample with a dilution series of positive controls, in which a known amount of antibody is combined with known concentrations of purified laminin 332 γ2 processed region polypeptide. Standard values obtained from normal samples may be compared with values obtained from samples from subjects suspected of having SCC. Deviation between standard and subject values establishes the presence of or predisposition to the disease state.

In other embodiments, the expression levels of genes are determined for different cellular states in the SCC phenotype; that is, the expression levels of genes in normal tissue and in SCC tissue are evaluated to provide expression profiles. An expression profile of a particular cell state or point of development is essentially a “fingerprint” of the state; while two states may have any particular gene similarly expressed, the evaluation of a number of genes simultaneously allows the generation of a gene expression profile that is unique to the state of the cell. By comparing expression profiles of cells in different states, information regarding which genes are important (including both up- and down-regulation of genes) in each of these states is obtained. Then, diagnosis may be done or confirmed: does tissue from a particular patient have the gene expression profile of normal or SCC tissue.

“Differential expression,” or grammatical equivalents as used herein, refers to both qualitative as well as quantitative differences in the genes' temporal and/or cellular expression patterns within and among the cells. Thus, a differentially expressed gene can qualitatively have its expression altered, including an activation or inactivation, in, for example, normal versus lymphoma tissue. That is, genes may be turned on or turned off in a particular state, relative to another state. As is apparent to the skilled artisan, any comparison of two or more states can be made. Such a qualitatively regulated gene will exhibit an expression pattern within a state or cell type which is detectable by standard techniques in one such state or cell type, but is not detectable in both. Alternatively, the determination is quantitative in that expression is increased or decreased; that is, the expression of the gene is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays, Lockhart, Nature Biotechnology, 14:1675-1680 (1996), hereby expressly incorporated by reference. Other techniques include, but are not limited to, quantitative reverse transcriptase PCR, Northern analysis and RNase protection. As outlined above, preferably the change in expression (i.e. upregulation or downregulation) is at least about 50%, more preferably at least about 100%, more preferably at least about 150%, more preferably, at least about 200%, with from 300 to at least 1000% being especially preferred.

As will be appreciated by those in the art, this may be done by evaluation at either the gene transcript, or the protein level; that is, the amount of gene expression may be monitored using nucleic acid probes to the DNA or RNA equivalent of the gene transcript, and the quantification of gene expression levels, or, alternatively, the final gene product itself (protein) can be monitored, for example through the use of antibodies to the laminin 332 γ2 domain IV and/or domain V peptides and standard immunoassays (ELISAs, etc.) or other techniques, including mass spectroscopy assays, 2D gel electrophoresis assays, etc. Thus, the proteins corresponding to laminin 332 γ2 processed region genes, i.e. those identified as being important in a SCC phenotype, can be evaluated in a SCC diagnostic test.

Numerous methods known to those of ordinary skill in the art find use in diagnosing SCC. For example, in some embodiments, proteins can be obtained from a sample or a patient are separated by electrophoresis on a gel (typically a denaturing and reducing protein gel, but may be any other type of gel including isoelectric focusing gels and the like). Following separation of the proteins, laminin 332 γ2 processed region polypeptide can be detected by immunoblotting with antibodies raised against the laminin 332 γ2 processed region polypeptide. Methods of immunoblotting are well known to those of ordinary skill in the art.

In some embodiments, antibodies find use in in situ imaging techniques. In this method cells are contacted with from one to many antibodies to laminin 332 γ2 processed region polypeptide. Following washing to remove non-specific antibody binding, the presence of the antibody or antibodies is detected. In one embodiment the antibody is detected by incubating with a secondary antibody that contains a detectable label. In another method the primary antibody to the laminin 332 γ2 processed region polypeptides contains a detectable label. In another preferred embodiment each one of multiple primary antibodies contains a distinct and detectable label. This method finds particular use in simultaneous screening for a plurality of laminin 332 γ2 processed region polypeptides. As will be appreciated by one of ordinary skill in the art, numerous other histological imaging techniques are useful in the invention.

In some embodiments the label is detected in a fluorometer which has the ability to detect and distinguish emissions of different wavelengths. In addition, a fluorescence activated cell sorter (FACS) can be used in the method.

In some embodiments, in situ hybridization of labeled nucleic acid probes to tissue arrays is done. For example, arrays of tissue samples, including SCC tissue and/or normal tissue, are made. In situ hybridization as is known in the art can then be done.

It is understood that when comparing the expression fingerprints between an individual and a standard, the skilled artisan can make a diagnosis as well as a prognosis. It is further understood that the genes which indicate the diagnosis may differ from those which indicate the prognosis.

In a preferred embodiment, the laminin 332 γ2 processed region polypeptide, antibodies, nucleic acids, and cells containing laminin 332 γ2 processed region polypeptide sequences are used in prognosis assays. In some embodiments, gene expression profiles can be generated that correlate to SCC severity, in terms of long term prognosis. Again, this may be done on either a protein or gene level, with the use of genes being preferred. In some embodiments, probes are attached to solid supports for the detection and quantification of sequences in a tissue or patient. The assays proceed as outlined for diagnosis.

The efficacy of therapeutic agents, such as antibodies and/or other candidate drugs also can be determined using the diagnostic assays described above. As will be appreciated by a person of skill in the art, assays to determine the efficacy of a therapeutic agent require the establishment of baseline values. In some embodiments, this is accomplished by combining body fluids, tissue biopsies, or cell extracts taken from a patient with SCC prior to treatment with the candidate drug with one or more antibody(ies) to a laminin 332 γ2 processed region polypeptide under conditions suitable for complex formation. Such conditions are well known in the art. The amount of standard complex formation may be quantified by comparing levels of antibody-target complex in the normal sample with a dilution series of positive controls, in which a known amount of antibody is combined with known concentrations of purified laminin 332 γ2 domain IV and/or domain V peptide. Standard values obtained from a patient before treatment may be compared with values obtained from a patient after treatment. Deviation between standard and subject values establishes the efficacy of the drug.

### Screening Assays

In some embodiments, the laminin 332 γ2 processed region polypeptide, antibodies, nucleic acids, and cells containing the peptides are used in screening assays. For example, screens for agents that modulate the SCC phenotype can be run. This can be done by screening for modulators of gene expression or for modulators of protein activity at the individual gene or protein level or by evaluating the effect of drug candidates on a “gene expression profile”. In some embodiments, the expression profiles are used in conjunction with high throughput screening techniques to allow monitoring for expression profile genes after treatment with a candidate agent (see Zlokarnik, et al., 1998, Science, 279: 84-8).

“Modulation” includes both an increase and a decrease in gene expression or activity. The preferred amount of modulation will depend on the original change of the gene expression in normal versus tumor tissue, with changes of at least 10%, preferably 50%, more preferably 100-300%, and in some embodiments 300-1000% or greater. If a gene exhibits a 4 fold increase in tumor compared to normal tissue, a decrease of about four fold is desired; a 10 fold decrease in tumor compared to normal tissue gives a 10 fold increase in expression for a candidate agent is desired, etc.

As will be appreciated by those in the art, this may be done by evaluation at either the gene or the protein level; that is, the amount of gene expression may be monitored using nucleic acid probes and the quantification of gene expression levels, or, alternatively, the level of the gene product itself can be monitored, for example through the use of antibodies and standard immunoassays. Alternatively, binding and bioactivity assays with the protein may be done as outlined below.

In some embodiments, gene expression monitoring is done and a number of genes, i.e. an expression profile, are monitored simultaneously. If desired, multiple protein expression monitoring can be done as well. In embodiments monitoring multiple genes or proteins, the corresponding probes are immobilized to solid supports. It is understood that immobilization can occur by any means, including for example; by covalent attachment, by electrostatic immobilization, by attachment through a ligand/ligand interaction, by contact or by depositing on the surface. “Solid support” or “solid substrate” refers to any solid phase material upon which a laminin 332 γ2 domain IV and/or domain V peptide, or antibody is synthesized, attached, ligated or otherwise immobilized. A solid support may be composed of organic polymers such as polystyrene, polyethylene, polypropylene, polyfluoroethylene, polyethyleneoxy, and polyacrylamide, as well as co-polymers and grafts thereof. A solid support may also be inorganic, such as glass, silica, controlled-pore-glass (CPG), or reverse-phase silica. The configuration of a solid support may be in the form of beads, spheres, particles, granules, a gel, or a surface. Surfaces may be planar, substantially planar, or non-planar. Solid supports may be porous or non-porous, and may have swelling or non-swelling characteristics. A solid support may be configured in the form of a well, depression or other container, vessel, feature or location. A plurality of solid supports may be configured in an array at various locations, addressable for robotic delivery of reagents, or by detection means including scanning by laser illumination and confocal or deflective light gathering.

Generally, a candidate bioactive agent is added prior to analysis. The term “candidate bioactive agent” or “drug candidate” or grammatical equivalents as used herein describes any molecule, e.g., protein, oligopeptide, small organic or inorganic molecule, polysaccharide, polynucleotide, etc., to be tested for bioactive agents that are capable of directly or indirectly altering either the SCC phenotype, binding to and/or modulating the bioactivity of a laminin 332 γ2 domain IV and/or domain V peptide, or the expression of a laminin 332 γ2 domain IV and/or domain V peptide sequence. In a particularly preferred embodiment, the candidate agent suppresses a SCC phenotype, for example to a normal tissue fingerprint. Generally a plurality of assay mixtures are run in parallel with different agent concentrations to obtain a differential response to the various concentrations. Typically, one of these concentrations serves as a negative control, i.e., at zero concentration or below the level of detection.

In one aspect, a candidate agent will neutralize the effect of a laminin 332 γ2 domain IV and/or domain V peptide. By “neutralize” is meant that activity of a protein is either inhibited or counter acted against so as to have substantially no effect on a cell.

Candidate agents encompass numerous chemical classes, though typically they are organic or inorganic molecules, preferably small organic compounds having a molecular weight of more than 100 and less than about 2,500 daltons. Preferred small molecules are less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, proteins, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In assays for altering the expression profile of one or more laminin 332 γ2 domain IV and/or domain V coding sequences, after the candidate agent has been added and the cells allowed to incubate for some period of time, the sample containing the sequences to be analyzed is added to a solid support. If required, the sequence is prepared using known techniques. For example, the sample may be treated to lyse the cells, using known lysis buffers, electroporation, etc., with purification and/or amplification such as PCR occurring as needed, as will be appreciated by those in the art.

Generally, one of the assay components is labeled to provide a means of detecting the binding complex of interest. By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the nucleic acids, proteins and antibodies at any position. For example, the label should be capable of producing, either directly or indirectly, a detectable signal. The detectable moiety may be a radioisotope, such as ³H, ¹²C, ³²P, ³⁵⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the label may be employed, including those methods described by Hunter et al., 1962, Nature, 144: 945; David et al., 1974, Biochemistry, 13: 1014; Pain et al., 1981, J. Immunol. Meth., 40: 219; and Nygren, 1982, J. Histochem. and Cytochem., 30: 407. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. As known in the art, unbound labeled streptavidin is removed prior to analysis.

As will be appreciated by those in the art, these assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference.

A variety of hybridization conditions may be used in the present invention, including high, moderate and low stringency conditions as outlined above. The assays are generally run under stringency conditions which allows formation of the label probe hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents may be included in the assays. These include reagents like salts, buffers, neutral proteins, e.g. albumin, detergents, etc which may be used to facilitate optimal hybridization and detection, and/or reduce non-specific or background interactions. Also reagents that otherwise improve the efficiency of the assay, such as protease inhibitors, nuclease inhibitors, anti-microbial agents, etc., may be used, depending on the sample preparation methods and purity of the target. In addition, either solid phase or solution based (i.e., kinetic PCR) assays may be used.

Once the assay is run, the data is analyzed to determine the expression levels, and changes in expression levels as between states, of individual genes, or individual proteins, forming an expression profile.

In some embodiments, screening is done to alter the biological function of the expression product of an laminin 332 γ2 domain IV and/or domain V gene. Again, having identified the importance of a gene in a particular state, screening for agents that bind and/or modulate the biological activity of the gene product can be run as is more fully outlined below.

In some embodiments, screens are designed to first find candidate agents that can bind to laminin 332 γ2 domain IV and/or domain V peptides, and then these agents may be used in assays that evaluate the ability of the candidate agent to modulate the laminin 332 γ2 domain IV and/or domain V peptide activity and the SCC phenotype. As will be appreciated by those in the art, there are a number of different assays which may be run; binding assays and activity assays.

In some embodiments, binding assays are done. In general, purified or isolated laminin 332 γ2 domain IV and/or domain V peptides are used. The methods comprise combining a laminin 332 γ2 domain IV and/or domain V peptide and a candidate bioactive agent, and determining the binding of the candidate agent to the peptide. Generally, the laminin 332 γ2 domain IV and/or domain V peptide or the candidate agent is non-diffusably bound to a solid support having isolated sample receiving areas (e.g. a microtiter plate, an array, etc.). Microtiter plates and arrays are especially convenient because a large number of assays can be carried out simultaneously, using small amounts of reagents and samples. The particular manner of binding of the composition is not crucial so long as it is compatible with the reagents, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

In some embodiments, the laminin 332 γ2 domain IV and/or domain V peptide is bound to the support, and a candidate bioactive agent is added to the assay. Alternatively, the candidate agent is bound to the support and the laminin 332 γ2 domain IV and/or domain V peptides are added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the candidate bioactive agent to the laminin 332 γ2 domain IV and/or domain V peptides may be done in a number of ways. In a preferred embodiment, the candidate bioactive agent is labeled, and binding determined directly. For example, this may be done by attaching all or a portion of the peptides to a solid support, adding a labeled candidate agent (for example a fluorescent label), washing off excess reagent, and determining whether the label is present on the solid support. Various blocking and washing steps may be utilized as is known in the art.

In some embodiments, only one of the components is labeled. For example, the proteins (or proteinaceous candidate agents) may be labeled at tyrosine positions using ¹²⁵I, or with fluorophores. Alternatively, more than one component may be labeled with different labels; using ¹²⁵I for the proteins, for example, and a fluorophor for the candidate agents.

In some embodiments, the binding of the candidate bioactive agent is determined through the use of competitive binding assays. In this embodiment, the competitor is a binding moiety known to bind to the laminin 332 γ2 domain IV and/or domain V peptide, such as an antibody, peptide, binding partner, ligand, etc. Under certain circumstances, there may be competitive binding as between the bioactive agent and the binding moiety, with the binding moiety displacing the bioactive agent.

In some embodiments, the candidate bioactive agent is labeled. Either the candidate bioactive agent, or the competitor, or both, is added first to the protein for a time sufficient to allow binding, if present. Incubations may be performed at any temperature which facilitates optimal activity, typically between 4 and 40° C. Incubation periods are selected for optimum activity, but may also be optimized to facilitate rapid high through put screening. Typically between 0.1 and 1 hour will be sufficient. Excess reagent is generally removed or washed away. The second component is then added, and the presence or absence of the labeled component is followed, to indicate binding.

In some embodiments, the competitor is added first, followed by the candidate bioactive agent. Displacement of the competitor is an indication that the candidate bioactive agent is binding to the laminin 332 γ2 domain IV and/or domain V peptide and thus is capable of binding to, and potentially modulating, the activity of the laminin 332 γ2 domain IV and/or domain V peptide. In this embodiment, either component can be labeled. Thus, for example, if the competitor is labeled, the presence of label in the wash solution indicates displacement by the agent. Alternatively, if the candidate bioactive agent is labeled, the presence of the label on the support indicates displacement.

In other embodiments, the candidate bioactive agent is added first, with incubation and washing, followed by the competitor. The absence of binding by the competitor may indicate that the bioactive agent is bound to the laminin 332 γ2 domain IV and/or domain V peptide with a higher affinity. Thus, if the candidate bioactive agent is labeled, the presence of the label on the support, coupled with a lack of competitor binding, may indicate that the candidate agent is capable of binding to the laminin 332 γ2 domain IV and/or domain V peptide.

In some embodiments, the methods comprise differential screening to identity bioactive agents that are capable of modulating the activity of the laminin 332 γ2 domain IV and/or domain V peptides. In this embodiment, the methods comprise combining a laminin 332 γ2 domain IV and/or domain V peptide and a competitor in a first sample. A second sample comprises a candidate bioactive agent, a laminin 332 γ2 domain IV and/or domain V peptide and a competitor. The binding of the competitor is determined for both samples, and a change, or difference in binding between the two samples indicates the presence of an agent capable of binding to the laminin 332 γ2 domain IV and/or domain V peptide and potentially modulating its activity. That is, if the binding of the competitor is different in the second sample relative to the first sample, the agent is capable of binding to the laminin 332 γ2 domain IV and/or domain V peptide.

In some embodiments, methods for screening for bioactive agents capable of modulating the activity of a laminin 332 γ2 domain IV and/or domain V peptide in a cell are provided. The methods comprise adding a candidate bioactive agent, as defined above, to a cell comprising laminin 332 γ2 domain IV and/or domain V peptides. Typically, primary human keratinocytes are used. The cells can also contain recombinant nucleic acids that encode laminin 332 γ2 domain IV and/or domain V peptide sequences, Ras and a stable NF-κB repressor mutant of IκBα (i.e. IKB) (see Dajee et al., 2003, Nature, 421: 630-643). Methods for culturing cells and for assaying cell scattering, adhesion and migration are described in Russell et al., 2003, J. Cell Sci., 116: 3543-3556, the entire contents of which are incorporated herein by reference.

In some embodiments, candidate agents can be introduced into immunodeficient mice that can subsequently be challenged with a laminin 332 γ2 domain IV and/or domain V peptide and monitored for the development of tumors. For example, intraperitoneal injections of antibodies against one or more laminin 332 γ2 domain IV and/or domain V peptides can be given to mice bearing human foreskin xenografts (see Examples; and Li et al., 2003, EMBO J., 22: 2400-2410). The mice can then be challenged with Ras/IKB transformed human keratinocytes and monitored for tumor growth and histology as described in Dajee et al., 2003, Nature, 421: 630-643.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

In one aspect, the assays are evaluated in the presence or absence or previous or subsequent exposure of physiological signals, for example hormones, antibodies, peptides, antigens, cytokines, growth factors, action potentials, pharmacological agents including chemotherapeutics, radiation, carcinogenics, or other cells (i.e. cell-cell contacts). In another example, the determinations are determined at different stages of the cell cycle process.

### Pharmaceutical Compositions

Bioactive agents having pharmacological activity are identified as described above. By “pharmacological activity” herein is meant that the compounds are able to enhance or interfere with the activity of laminin 332 γ2 domain IV and/or domain V peptides. The compounds having the desired pharmacological activity may be administered in a physiologically acceptable carrier to a patient. A “patient” includes both humans and other animals, particularly mammals, and domestic animals. Thus, the methods are applicable to both human therapy and veterinary applications.

In some embodiments, bioactive agents include antibodies that recognize laminin 332 γ2 domain IV and/or domain V peptides and that have been demonstrated to inhibit or modulate SCC as described herein. In other embodiments, bioactive agents include antisense compositions. These agents can be delivered directly or in pharmaceutical compositions along with suitable carriers or excipients, as well known in the art. Present methods of treatment include embodiments providing for administration of an effective amount of a compound or agent that inhibits the activity or expression of a laminin 332 γ2 domain IV and/or domain V peptides to a patient in need of treatment.

An effective amount of such agents can readily be determined by routine experimentation, as can the most effective and convenient route of administration and the most appropriate formulation. Various formulations and drug delivery systems are available in the art. (See, e.g., Remington's Pharmaceutical Sciences, supra.)

Suitable routes of administration may, for example, include oral, rectal, transmucosal, transdermal, nasal, or intestinal administration and parenteral delivery, including intramuscular, subcutaneous, intramedullary injections, as well as intrathecal, direct intraventricular, intravenous, intraperitoneal, intranasal, or intraocular injections. The agent or composition thereof may be administered in a local rather than a systemic manner. For example, a suitable agent can be delivered via injection or in a targeted drug delivery system, such as a depot or sustained release formulation.

The pharmaceutical compositions may be manufactured by any of the methods well-known in the art, such as by conventional mixing, dissolving, granulating, dragee-making, levigating, emulsifying, encapsulating, entrapping, or lyophilizing processes. The compositions can include one or more physiologically acceptable carriers such as excipients and auxiliaries that facilitate processing of active molecules into preparations for pharmaceutical use. Proper formulation is dependent upon the route of administration chosen.

For example, for injection, the composition may be formulated in aqueous solutions, preferably in physiologically compatible buffers such as Hanks's solution, Ringer's solution, or physiological saline buffer. For transmucosal or nasal administration, penetrants appropriate to the barrier to be permeated are used in the formulation. Such penetrants are generally known in the art. For oral administration, the agents can be formulated readily by combining the active agents with pharmaceutically acceptable carriers well known in the art. Such carriers enable the agents of the invention to be formulated as tablets, pills, dragees, capsules, liquids, gels, syrups, slurries, suspensions and the like, for oral ingestion by a subject. The agents may also be formulated in rectal compositions such as suppositories or retention enemas, e.g., containing conventional suppository bases such as cocoa butter or other glycerides.

Pharmaceutical preparations for oral use can be obtained as solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Pharmaceutical preparations for oral administration include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration.

For administration by inhalation, the agents can be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide, or any other suitable gas. In the case of a pressurized aerosol, the appropriate dosage unit may be determined by providing a valve to deliver a metered amount. Capsules and cartridges for use in an inhaler or insufflator may be formulated. These typically contain a powder mix of the agent and a suitable powder base such as lactose or starch.

Compositions formulated for parenteral administration by injection, e.g., by bolus injection or continuous infusion can be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Formulations for parenteral administration include aqueous solutions of the compound or agent to be administered, including in water-soluble form.

Suspensions of the active agents may also be prepared as appropriate oily injection suspensions. Suitable lipophilic solvents or vehicles include fatty oils such as sesame oil and synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the agents to allow for the preparation of highly concentrated solutions. Alternatively, the active ingredient may be in powder form for constitution with a suitable vehicle, e.g., sterile pyrogen-free water, before use.

As mentioned above, the compositions can also be formulated as a depot preparation. Such long acting formulations may be administered by implantation (for example, subcutaneously or intramuscularly) or by intramuscular injection. Thus, for example, the present agents may be formulated with suitable polymeric or hydrophobic materials (for example as an emulsion in an acceptable oil) or ion exchange resins, or as sparingly soluble derivatives, for example, as a sparingly soluble salt.

Suitable carriers for the hydrophobic molecules of the invention are well-known in the art and include co-solvent systems comprising, for example, benzyl alcohol, a nonpolar surfactant, a water-miscible organic polymer, and an aqueous phase. The co-solvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system is effective in dissolving hydrophobic agents and produces low toxicity upon systemic administration. Naturally, the proportions of a co-solvent system may be varied considerably without destroying its solubility and toxicity characteristics. Furthermore, the identity of the co-solvent components may be varied. For example, other low-toxicity nonpolar surfactants may be used instead of polysorbate 80, the fraction size of polyethylene glycol may be varied, other biocompatible polymers may replace polyethylene glycol, e.g. polyvinyl pyrrolidone, and other sugars or polysaccharides may substitute for dextrose.

Alternatively, other delivery systems for hydrophobic molecules may be employed. Liposomes and emulsions are well known examples of delivery vehicles or carriers for hydrophobic drugs. Liposomal delivery systems are discussed above in the context of gene-delivery systems. Certain organic solvents such as dimethylsulfoxide also may be employed, although usually at the cost of greater toxicity. Additionally, the agents may be delivered using sustained-release systems, such as semi-permeable matrices of solid hydrophobic polymers containing the effective amount of the composition to be administered. Various sustained-release materials are established and available to those of skill in the art. Sustained-release capsules may, depending on their chemical nature, release the agents for a few weeks up to over 100 days. Depending on the chemical nature and the biological stability of the therapeutic reagent, additional strategies for protein stabilization may be employed.

For any composition employed herein, a therapeutically effective dose can be estimated initially using a variety of techniques well-known in the art. For example, in a cell culture assay, a dose can be formulated in animal models to achieve a circulating concentration range that includes the IC₅₀ as determined in cell culture. Where inhibition of human laminin 5 alpha 3 G4-5 domain activity is desired, the concentration of the test agent that achieves a half-maximal inhibition of activity can be determined. Dosage ranges appropriate for human subjects can be determined, using data obtained from cell culture assays and other animal studies.

A therapeutically effective dose of an agent refers to that amount of the agent that results in amelioration of symptoms or a prolongation of survival in a subject. Toxicity and therapeutic efficacy of such molecules can be determined by standard pharmaceutical procedures in cell cultures or experimental animals, e.g., by determining the LD.sub.50 (the dose lethal to 50% of the population) and the ED₅₀ (the dose therapeutically effective in 50% of the population). The dose ratio of toxic to therapeutic effects is the therapeutic index, which can be expressed as the ratio LD₅₀/ED₅₀. Agents that exhibit high therapeutic indices are preferred.

Dosages preferably fall within a range of circulating concentrations that includes the ED₅₀ with little or no toxicity. Dosages may vary within this range depending upon the dosage form employed and the route of administration utilized. The exact formulation, route of administration, and dosage should be chosen, according to methods known in the art, in view of the specifics of a subject's condition.

Dosage amount and interval may be adjusted individually to provide plasma levels or tissue levels of the active moiety which are sufficient to affect the expression or activity of laminin 332 γ2 domain IV and/or domain V peptides, as desired, i.e. minimal effective concentration (MEC). The MEC will vary for each agent but can be estimated from, for example, in vitro data, such as the concentration necessary to achieve 50-90% inhibition of activity using the assays described herein. Dosages necessary to achieve the MEC will depend on individual characteristics and route of administration. Agents or compositions thereof should be administered using a regimen which maintains plasma levels above the MEC for about 10-90% of the duration of treatment, preferably about 30-90% of the duration of treatment, and most preferably between 50-90%. In cases of local administration or selective uptake, the effective local concentration of the drug may not be related to plasma concentration.

The amount of agent or composition administered will, of course, be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack. The pack or dispenser device may be accompanied by instructions for administration: Compositions comprising a agent of the invention formulated in a compatible pharmaceutical carrier may also be prepared, placed in an appropriate container, and labeled for treatment of an indicated condition. Suitable conditions indicated on the label may include treatment of disorders or diseases, such as squamous cell carcinoma or other cancers and conditions associated with altered expression of laminin 332 γ2 domain IV and/or domain V peptides.

### EXAMPLES

### Introduction

Squamous cell carcinoma (SCC) is a highly prevalent malignant epithelial neoplasm which causes considerable morbidity and mortality throughout the world. Laminin-332 (formerly termed laminin-5) is an extracellular basement membrane zone (BMZ) molecule consisting of α3, β3 and γ2 subunits. Laminin-332 is synthesized and deposited at the invasive front of many tumors, especially SCC, and its expression correlates with invasiveness in a number of types of SCC tumors (see Holler (2005) Breast Cancer Res 7, 166-167). Increased laminin-332 expression correlates with a poor prognosis for patients with oral SCC (Nordemar, et al. (2001) Anticancer Res 21, 509-512; Yamamoto et al. (2001) Clin Cancer Res 7, 896-900). In addition, laminin-332 expression has been shown to be required for tumor formation in a well characterized in vivo model of human SCC (Dajee et al. (2003) Nature 421, 639-643). These collective findings suggest a critical role for laminin-332 in SCC carcinogenesis.

Laminin-332 undergoes processing of its a3 chain and γ2 chains, and laminin-332 exists in a fully processed state in normal mature epithelial BMZs. The domains on the laminin α3 and γ2 chains are proteolytically removed and share similar functions, including binding heparan and promoting laminin-332 deposition (Gagnoux-Palacios et al. (2001) J Cell Biol 153, 835-850; Sigle et al. (2004) J Cell Sci 117, 4481-4494).

The data provided herein demonstrates that the processed domains on laminin-332 plays a critical role in SCC tumor formation but not in normal tissue adhesion.

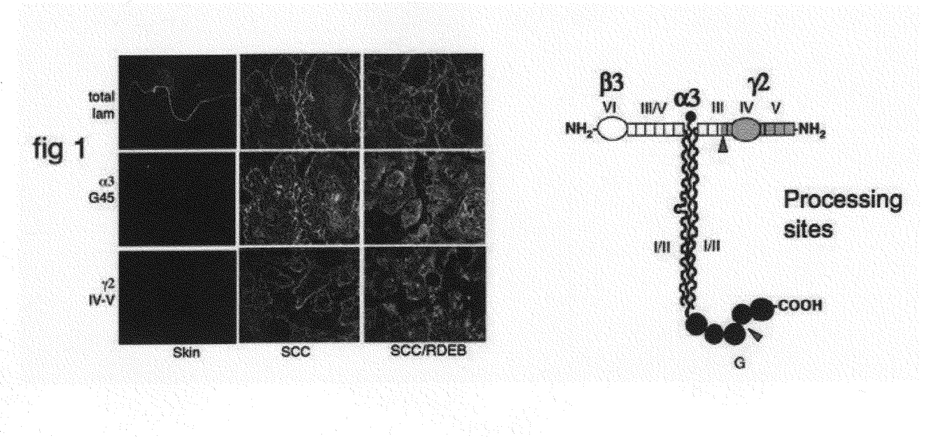

Proteolytic fragments of laminin-332 plays a role in SCC tumorigenesis: Laminin-332 undergoes processing of its α3 chain and γ2 chains which removes 50 and 35 kDa fragments (representing the γ2 domain IV-V and the α3 G45 domain respectively) from the laminin trimer. Using antibodies against these fragments we showed a complete lack of staining in normal skin, but positive expression in frozen sections of SCC tumors in two patient SCC tumors by IDIF (FIG. 1). On the right of the figure we show the processing sites for each of these laminin chains and the small fragments which are liberated after the cleavage events are the domains recognized by these antibodies.

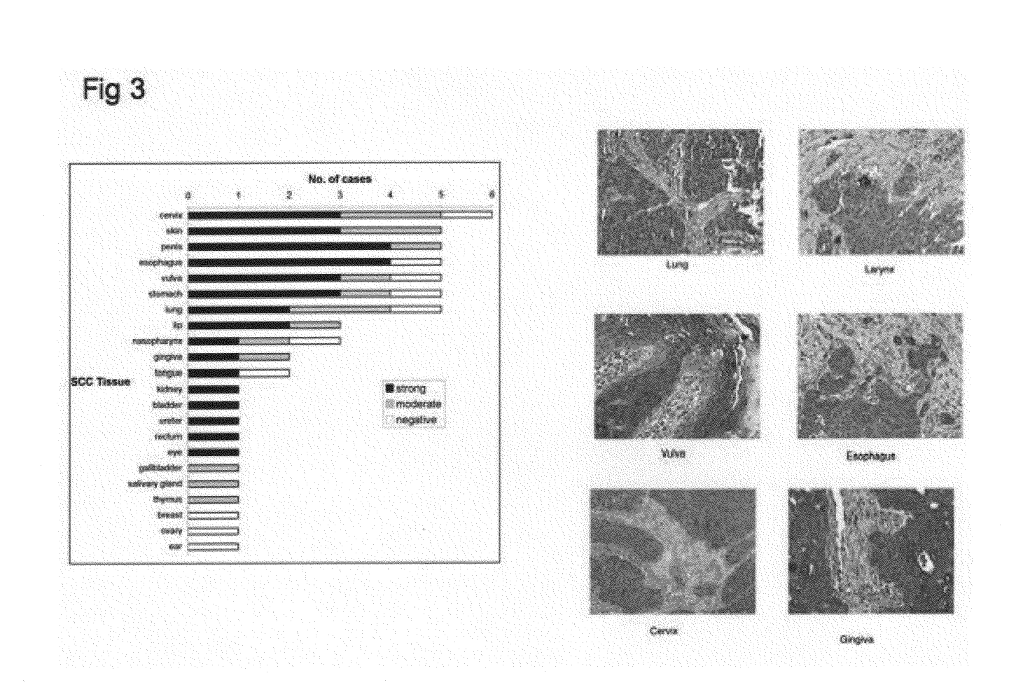

The G45 pAb and a polyclonal antibody against domain IV of the processed proteolytic fragment of the laminin γ2 chain have each has proven amenable to antigen retrieval methods and through this technique we analyzed two tissue microarrays of SCC tumors derived from a number of tissues (Biomax), and found moderate to strong expression of the G45 domain in over 75% of the samples analyzed (FIG. 2 for G45 chain antibody and FIG. 3 for domain IV γ2 chain antibody).

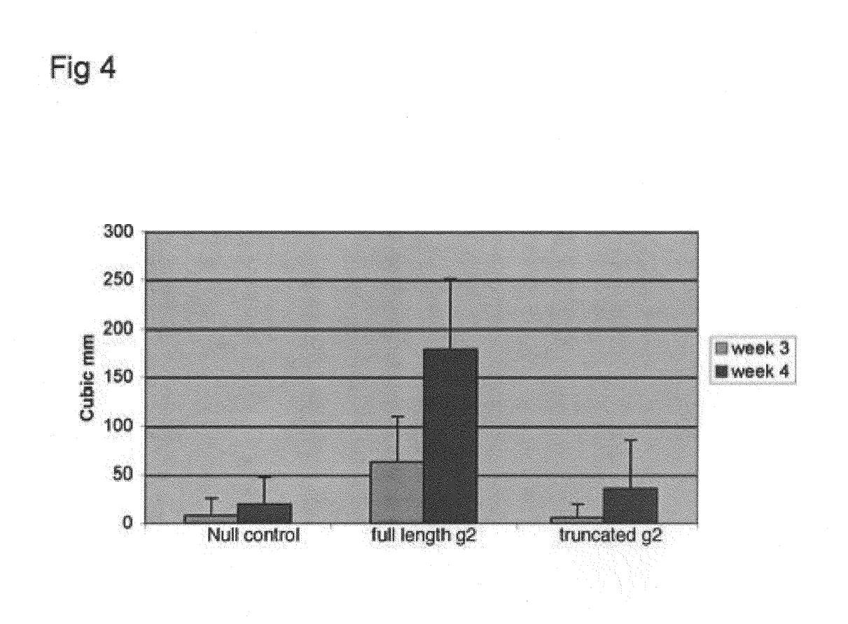

Thus, in contrast to normal tissues, SCC tumors contain a significant amount of unprocessed laminin-332. In addition to a full length wild type γ2 cDNA, we produced a truncated laminin γ2 cDNA which created a protein product lacking the first 434 residues on the N-terminus (domain IV-V) proximal to where normal processing occurs (Sasaki et al. (2001) J Mol Biol 314, 751-763). We retrovirally expressed these constructs into primary laminin γ2 chain null junctional epidermolysis bullosa keratinocytes. Like wild type γ2 constructs the truncated gamma2 construct produced expression of fully assembled trimeric laminin-332 by JEB keratinocytes. Following transformation of these cells with Ras/IKBA in a well characterized model of human SCC tumorigenesis. we next analyzed the ability of transformed cells to produce human SCC-like tumors after subcutaneous injection to immunodeficient mice (FIG. 4). Transformed cells lacking domain IV-V showed a marked impairment of tumor formation.

### Example 2

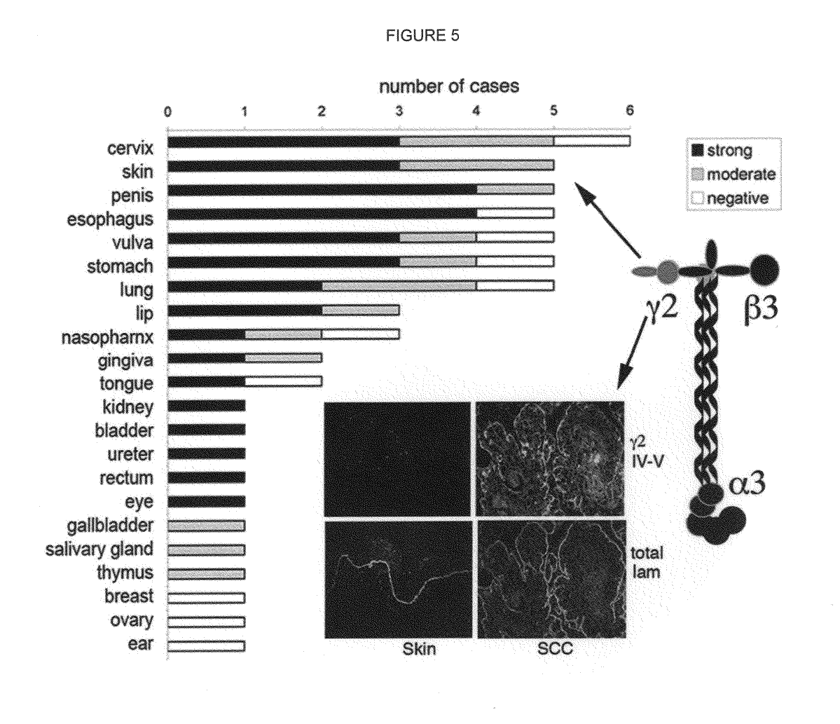

Precursor domain of laminin γ2 chain plays a key role in SCC tumorigenesis. Like its α3 chain described previously, laminin-332 also undergoes processing of its γ2 chain which removes a 50 kDa fragment (representing the γ2 domain IV-V, shown in red in schematic, FIG. 5). Using antibodies against this fragment we showed a complete lack of staining in normal skin, but positive expression in SCC tumors by IDIF as well as in a tissue microarray of SCC tumors derived from a wide range of tissues (Biomax). Overall, we found moderate to strong expression of the γ2 domain IV-V in over 75% of the samples analyzed.

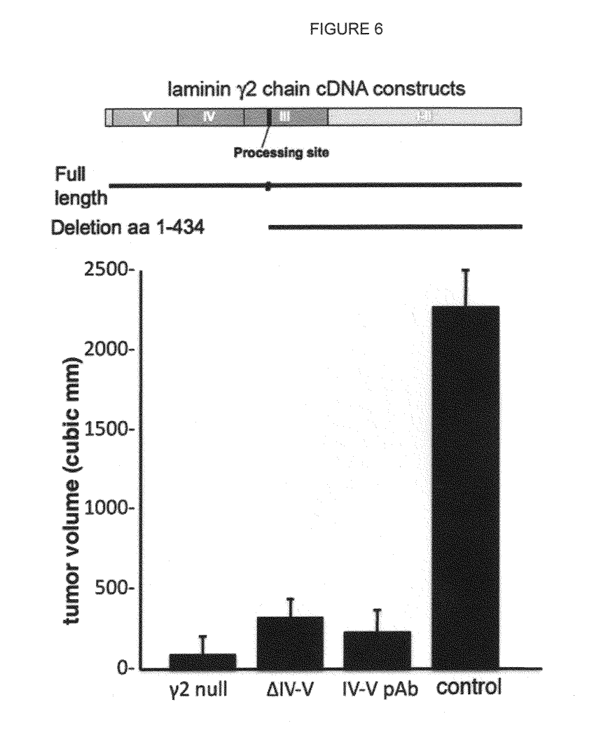

We then produced a truncation mutant of the laminin γ2 chain which contains a deletion of the precursor domain IV-V (ΔIV-V) which is normally proteolytically removed (aa 1-434) (FIG. 6). This mutant was well as wild type laminin γ2 cDNA was retrovirally introduced into γ2 null keratinocytes derived from a patient with junctional epidermolysis bullosa with null mutations of the LAMC2 gene.

After Ras, IκBα transformation, these cells were subcutaneously transferred to nude mice and analyzed for their ability to form tumors. We used an affinity purified polyclonal antibody produced against the laminin γ2 domain IV-V. Mice were injected with this antibody (IV-V pAb) or control antibody once a week for five weeks (0.5 mg/mouse/wk).

As seen in FIG. 6, either truncation of the precursor domains IV-V of the laminin γ2 chain or injection of ant-γ2 IV-V pAb resulted in a significant impairment of tumor formation after 5 weeks. Each condition shown was the average of 5 mice tested. No blistering or other abnormalities were seen on careful histologic or EM analysis of all mice treated with the γ2 pAb. This suggests that the precursor γ2 domain of laminin-332 also plays a critical role in SCC tumorigenesis.

As the laminin γ2 precursor domain of pro-laminin-332 is highly expressed in SCC, but not normal tissues, and plays a critical role in human SCC tumorigenesis in vivo, it represents an excellent target for anti-cancer therapy.

### Example 3

### Assays for Detecting Inhibition of SCC Tumorigenesis

At present, it is unclear whether domain IV or domain V sequences performs its function in SCC before or after it becomes processed and dissociated from laminin-332. This is tested in restoring tumor generating capabilities in Ras/IKB transformed 1337Tr keratinocytes by adding exogenous G4-5 protein or G4-5 cDNA. If Ras/IKB transformed 1337Tr keratinocytes can form tumors in nude mice after receiving domain IV or domain V sequences protein or cDNA, this would indicate that the domain is active in SCC tumors in a soluble form.

The effects of anti laminin γ2 domain IV or domain V sequences antibodies on tumor development is tested. Sufficient antibody is injected to maintain a circulating titer of 1:1000 as tested by dilution of mouse sera by Western blot analysis protein. Laminin γ2 domains cloned into pGEX vector are utilized to produce domain-specific bacterial fusion proteins. Proteins are affinity purified on a GST column, and GST tags are subsequently removed by enterokinase (Invitrogen). Isolated domain proteins will then be used to produce rabbit polyclonal antisera at Josman Labs, Napa, Calif., according to their recommended protocols.

Additional domain specific protein is produced, affinity purified and coupled to a Sepharose CL-4B column at a concentration of 0.5 mg protein per ml of gel. Polyclonal antisera will be affinity purified on a peptide conjugated-sepharose columns. Affinity purified antibodies are dialyzed into PBS and filter sterilized. Aantibodies are tested by IP injection of immunodeficient mice bearing human foreskin xenografts by a technique which we have utilized previously (Li et al., 2003, EMBO J., 22:2400-2410). Titers of circulating antibodies in treated mice are assessed at 3 day intervals using sera obtained from tail vein bleeds. The amount of antibody injected and the injection intervals is adjusted to maintain a titer sufficient to detect laminin protein by Western blot at a 1:1000 serum dilution. We will clinically assess foreskin grafts and mouse skin over the course of three weeks of injections to determine whether epidermal separation is noted, and mice will be examined by autopsy to detect any epithelial sloughing of mucosa or internal organs.

Using the proper antibody dose and injection intervals, antibody inhibition of Ras/IKB wild type keratinocyte derived tumors is performed. In these studies, nude mice are administered periodic antibody injections to maintain a constant circulating antibody titer as described above. Once antibody titers are initiated, then mice will receive SC injections of Ras/IKB transformed human keratinocytes. Three groups of 6 mice each will be studied, using affinity purified anti laminin γ2 antibody, or mouse IgG. G domain antibody conditions will be analyzed for tumor growth and tumor histology as previously described (Dajee et al., 2003, Nature, 421:639-43).

While the foregoing has presented specific embodiments, it is to be understood that these embodiments have been presented by way of example only. It is expected that others will perceive and practice variations which, though differing from the foregoing, do not depart form the spirit and scope of the inventions as described and claimed herein. All patent applications, patents, and literature references cited in this specification are hereby incorporated by reference in their entirety. In case of conflict or inconsistency, the present description, including definitions, will control.

What is claimed is:

1. A composition for treating squamous cell carcinoma (SCC) comprising an antibody that specifically binds a laminin 332 γ2 processed region polypeptide, and a pharmaceutically acceptable carrier.

2. A composition according to claim 1, comprising an antibody specific for SEQ ID NO:2, residues 1-434.

3. A composition according to claim 2, wherein said antibody binds to a sequence within domain IV.

4. A composition according to claim 2, wherein said antibody binds to a sequence within domain V.

5. A composition according to claim 2, wherein said antibody is a polyclonal antibody.

6. A composition according to claim 2, wherein said antibody is a monoclonal antibody.

7. A composition according to claim 2, wherein said SCC is selected from the group consisting of skin cancer, lung cancer, head cancer, gastric cancer, colorectal, throat cancer, cancer of the urinary tract, cancer of the reproductive tract, esophageal cancer, and bronchiogenic carcinoma.

8. A method of treating squamous cell carcinoma (SCC) in a patient comprising administering a therapeutically effective amount of one or more antibodies in a pharmaceutically acceptable carrier, wherein one or more of said antibodies is capable of specifically binding a laminin 332 γ2 processed region polypeptide, and inhibiting SCC tumorigenesis.

9. The method according to claim 8, wherein the one or more antibodies is specific for SEQ ID NO:2, residues 1-434.

10. The method according to claim 9, wherein said antibody is a polyclonal antibody.

11. The method according to claim 9, wherein said antibody is a monoclonal antibody.

12. The method according to claim 9, wherein said SCC is selected from the group consisting of skin cancer, lung cancer, head cancer, gastric cancer, colorectal, throat cancer, cancer of the urinary tract, cancer of the reproductive tract, esophageal cancer, and bronchiogenic carcinoma.

13. A method for diagnosing the presence of SCC comprising the steps of: a) contacting a sample suspected of comprising neoplastic epithelial cells with an antibody capable of specifically binding a laminin 332 γ2 processed region polypeptide, b) detecting the binding of said antibody; and, c) diagnosing therefrom the presence or absence of SCC in said sample.

14. The method according to claim 13, wherein the antibody is specific for SEQ ID NO:2, residues 1434.

15. A method according to claim 14, wherein said antibody further comprises a detectable label.

16. The method according to claim 14, wherein said epithelial cells are selected from the group consisting of squamous cells, keratinocytes, mucosal epithelial cells, gastrointestinal epithelial cells, corneal epithelia of the eye, and epithelial cells of the urinary and reproductive tract.

17. The method according to claim 14, wherein said sample is a tissue sample.

18. The method according to claim 14, wherein said sample is a urine sample.

19. The method according to claim 14, wherein said sample is a blood sample.

20. A method of screening for candidate agents that inhibit SCC tumorigenesis comprising the steps of:
identifying a candidate binding agent capable of binding a γ2 processed region polypeptide;
contacting SCC tumor cells with said agent under physiological conditions and determining the ability of the tumor cells to form tumors.

21. The method according to claim 20, wherein said candidate binding agent is selected from the group consisting of antibodies and fragments thereof, small molecules, polypeptides, and aptamers.

22. The method according to claim 20, wherein said tumor cells are provided in vitro culture conditions.

23. The method according to claim 20, wherein the tumor cells are provided in vivo.
